(19) United States
(12) Patent Application Publication
(10) Pub. No.: US 2024/0066187 A1
(43) Pub. Date: Feb. 29, 2024

(54) RADIOPAQUE LINED HEAT SHRINKABLE TUBING

(71) Applicant: ZEUS COMPANY INC., ORANGEBURG, SC (US)

(72) Inventors: Brian R. Tomblin, Columbia, SC (US); Matthew Cox, St. Matthews, SC (US); Shannon M. Giovannini, Columbia, SC (US); Jacob Coleman, Lexington, SC (US); Parastoo Azamian, West Columbia, SC (US)

(21) Appl. No.: 18/238,160

(22) Filed: Aug. 25, 2023

Related U.S. Application Data
(60) Provisional application No. 63/400,807, filed on Aug. 25, 2022.

(51) Int. Cl. A61L 29/18; A61L 29/04; A61L 29/12; C08L 27/18; C08L 77/00

(52) U.S. Cl. CPC A61L 29/18; A61L 29/041; A61L 29/126; C08L 27/18; C08L 77/00; C08L 2203/02; C08L 2203/18

(57) ABSTRACT

A radiopaque lined heat shrinkable tubing is disclosed. A non-limiting example of a radiopaque lined heat shrinkable tubing is a multilayer construction having an inner layer and an outer layer, wherein the inner layer includes a thermoplastic that is highly loaded with a radiopaque filler, and the outer layer includes a fluoropolymer heat shrink tube.

### CROSS-REFERENCE TO RELATED APPLICATIONS

The present application claims the benefit of U.S. Provisional Patent Application No. 63,400,807, filed Aug. 25, 2022, the disclosure of which is incorporated herein by reference in its entirety.

### FIELD

The present application is directed to heat shrink tubing and methods for making such heat shrink tubing, which finds application in a variety of fields.

### BACKGROUND

Heat shrinkable tubing (also referred to herein as heat shrink tubing) generally comprises a plastic material that is extruded into a tubular form and expanded. The extruded and expanded tube is designed to shrink (e.g., decrease in diameter) when heated to a given temperature. As such, heat shrink tubing can serve various functions. It can provide a tight, protective jacketing to closely cover and insulate various elements (e.g., to protect them from abrasion and to provide thermal, chemical, moisture, and/or electrical insulation); it can serve to bundle certain elements together (i.e., within the same heat shrink tube); it can serve to seal isolate certain elements from others; it can be used to join/fuse two elements, e.g., two tubes together; and it can serve to modify the properties of an underlying material (e.g., by closing around another material and shrinking that material as well). These capabilities render heat shrink tubing useful for various purposes and heat shrink tubing finds use across various fields, e.g., medical, chemical, electrical, optical, electronic, aerospace, automotive, and telecommunications fields.

In the medical context, heat shrink tubing is particularly beneficial in designing increasingly small and more complex devices to be inserted into the body, including catheters, endoscopes, etc. One representative medical use of heat shrink tubing is in the context of manufacturing a guide catheter, comprising a tubular structure having an inner layer of a polymer, a middle layer of a wire braid and an outer layer of another polymer. To assemble such catheters, a heat shrink tube is typically applied to an assembled shaft around a mandrel and the assembly is exposed to high temperature sufficient to shrink the heat shrink tube. Under these conditions, the outer polymeric layers within the catheter melt and flow, and the heat shrink tube contracts, providing compressive forces such that the inner and outer polymeric layers of the catheter shaft can bond together, encapsulating the wire braid within. The heat shrink tubing may then be removed and discarded and the catheter assembly is removed from the mandrel. See, e.g., the disclosures of U.S. Pat. No. 7,306,585 to Ross and U.S. Pat. No. 5,755,704 to Lunn, which are incorporated herein by reference.

Some medical devices comprise radiopaque markings to facilitate better precision during medical procedures. The radiopaque markings are very visible on x-rays. For example, the radiopaque markings cause a sharp contrast to become visible on the x-rays to delineate radiopaque features of the medical device and non-radiopaque features of the medical device or of the patient body. Accordingly, medical practitioners can use the sharp contrast caused by radiopaque markings to assist in guiding the medical device through the patient's body.

Medical devices have been developed which use radiopaque markings that are applied to a catheter shaft, such as a marker band applied to an outside layer of the catheter shaft. For example, traditional marker bands comprise a short, thin-walled tube machined from gold or platinum that is mechanically crimped or swaged at specific locations on a catheter. However, use of such radiopaque markings has led to the radiopaque markings on medical devices to come loose, shift in place along the medical device (e.g., the catheter shaft), or fall off. Such outcomes have negative implications for the medical procedure, ranging from imprecise positioning of the medical device to a negative patient outcome from a dislodged marker band.

### SUMMARY

The present disclosure relates to tubes, and in particular, heat shrink tubes, comprising a radiopaque component. Such heat shrink tubes are described herein in expanded (“heat shrink”) form and in processed (“shrunk”) form (e.g., as a component of a construction such as a catheter construction). Certain multi-layered heat shrink tubes are described herein also as a means for introducing just one layer thereof within the constructions, e.g., such that one or more layers of the processed heat shrink tube are removed after placement of the desired layer.

The disclosure includes, without limitation, the following embodiments:

Embodiment 1: A tube, comprising: an inner layer comprising thermoplastic material, wherein the thermoplastic is loaded with a radiopaque material; and an outer layer comprising a fluoropolymeric material.

Embodiment 2: The tube of Embodiment 1, wherein the thermoplastic material comprises one or more of Polyamide or Pebax (Poly-ether-block-amide).

Embodiment 3: The tube of Embodiment 1 or 2, wherein the inner layer comprises the radiopaque material in an amount of about 5% to about 80% by weight, based on a total weight of the inner layer.

Embodiment 4: The tube of Embodiment 1 or 2, wherein the inner layer comprises the radiopaque material in an amount of about 25% to about 50% by weight, based on a total weight of the inner layer.

Embodiment 5: The tube of Embodiment 1 or 2, wherein the inner layer comprises the radiopaque material in an amount of about 30% to about 40% by weight, based on a total weight of the inner layer.

Embodiment 6: The tube of any of Embodiments 1-5, wherein the radiopaque material is dispersed substantially uniformly throughout the inner layer.

Embodiment 7: The tube of any of Embodiments 1-6, wherein the outer layer is peelable.

Embodiment 8: The tube of any of Embodiment 1-7, wherein the radiopaque material comprises barium sulfate.

Embodiment 9: The tube of any of Embodiments 1-8, wherein the radiopaque material comprises one or more of bismuth oxychloride, bismuth subcarbonate and tungsten metal powder.

Embodiment 10: The tube of any of Embodiments 1-9, wherein the inner layer has an average wall thickness of about 0.010″.

Embodiment 11: The tube of any of Embodiments 1-10, wherein the inner layer has an average wall thickness between 0.005″ and 0.020″.

Embodiment 12: The tube of any of Embodiments 1-11, wherein the fluoropolymeric material of the outer layer comprises fluorinated ethylene propylene (FEP).

Embodiment 13: The tube of any of Embodiments 1-11, wherein the fluoropolymeric material of the outer layer comprises polytetrafluoroethylene (PTFE).

Embodiment 14: The tube of any of Embodiments 1-11, wherein the fluoropolymeric material of the outer layer comprises perfluoroalkoxy alkanes (PFA).

Embodiment 15: The tube of any of Embodiments 1-14, wherein the outer layer has a higher melt point than the inner layer.

Embodiment 16: The tube of any of Embodiments 1-14, wherein the inner and outer layers are in expanded form.

Embodiment 17: The tube of any of Embodiments 1-16, wherein a shrink ratio of an expanded inner diameter of the tube to a recovered inner diameter of the tube is greater than 1.2:1.

Embodiment 18: A medical device manufactured using the tube of any of Embodiments 1-17 (e.g., by positioning the tube and applying heat and/or pressure to shrink the tube in place).

Embodiment 19: The medical device of Embodiment 18, wherein the outer layer is removed (e.g., after positioning and applying heat and/or pressure to shrink the tube in place).

Embodiment 20: A medical device comprising a tube, wherein the tube comprises a thermoplastic material loaded with a radiopaque material.

Embodiment 21: The medical device of Embodiment 20, wherein the radiopaque material is dispersed substantially uniformly throughout the tube.

Embodiment 22: A medical device comprising the tube of any of Embodiments 1-17 (in expanded or shrunk form).

Embodiment 23: A medical device comprising at least the inner layer of the tube of any of Embodiments 1-17.

Embodiment 24: The medical device of any of Embodiments 18-23, wherein the medical device is a catheter.

Embodiment 25: The medical device of any of Embodiment 23 or 24, wherein the outer layer of the tube is removed during assembly of the medical device.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present invention will become apparent from the following.

### BRIEF DESCRIPTION OF THE DRAWINGS

In order to provide an understanding of the embodiments of the invention, reference is made to the appended drawings, which are not necessarily drawn to scale, and in which reference numerals refer to components of exemplary embodiments of the invention. The drawings are exemplary only and should not be construed as limiting the invention.

FIG. 1 is a general schematic of certain constructions 8 of the disclosure;

FIG. 2 is a general schematic of certain methods for forming constructions 8 of the disclosure; and

FIG. 3 is a general schematic of certain methods of placing/using certain constructions 8 of the disclosure, e.g., within a medical device (other components not shown).

### DETAILED DESCRIPTION

The present invention will now be described more fully hereinafter with reference to the accompanying figures, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The present disclosure relates to heat shrink tubes comprising radiopaque filler and to methods of making and using such tubes. The disclosure further provides constructions, e.g., tubings comprising two or more layers (in expanded and heat shrunk forms). The disclosure additionally provides for use of these constructions, which may, in some embodiments, provide for the placement and use of just one layer of the constructions, e.g., via removal of one or more layers of the constructions after placement. As such, the disclosure describes tubes/layers comprising radiopaque filler and their positioning/use within medical devices such as catheter shafts.

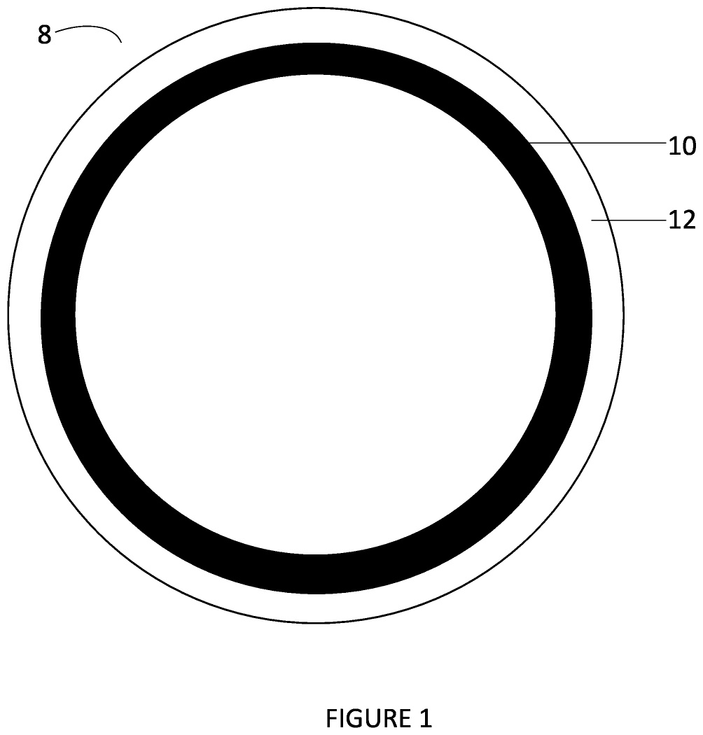

In one embodiment, a construction 8 is provided comprising a first, inner layer 10 and a second, outer layer 12, as schematically depicted in FIG. 1. The first, inner layer 10 comprises a thermoplastic material comprising one or more radiopaque materials; and the second, outer layer 12 comprises a fluoropolymer material.

The first, inner layer 10 generally comprises a thermoplastic polymer. Thermoplastic polymers are known and examples of suitable thermoplastic polymers include, but are not limited to, polyamides, Pebax (Poly-ether-block-amide), urethanes, polyethylene, or co-polymers, derivatives, or combinations thereof. Layer 10 can, in some embodiments, consist essentially of the thermoplastic polymer(s) and the radiopaque material.

The first, inner layer 10 further comprises one or more radiopaque materials. Radiopaque materials are generally understood to be materials that are opaque to x-rays, such that devices or components thereof containing such materials are visible under fluoroscopy or x-ray imaging. In some embodiments, radiopaque materials are dense metals, e.g., comprising tungsten metal, such as tungsten metal powder. In some embodiments, radiopaque materials are barium-containing compounds (e.g., barium sulfate) or bismuth-containing compounds (e.g., bismuth oxychloride or bismuth subcarbonate).

Radiopaque materials can be, for example, in the form of solid materials, e.g., powders. Advantageously, they are largely homogeneously dispersed throughout layer 10, but the disclosure is not limited thereto and layer 10 may comprise, in some embodiments, clumps or clusters of radiopaque materials. The amount (i.e., loading) of radiopaque material within a given layer 10 can vary and can be, for example, about 5% to about 80% by weight based on the weight of the layer 10, e.g., about 5% to about 60% by weight, about 5% to about 50% by weight, about 25% to about 60% by weight, about 25% to about 50% by weight, about 30% to about 60% by weight, about 30% to about 50% by weight, or about 30% to about 40% by weight based on the weight of the layer 10. Generally, although not limited thereto, barium and bismuth-based radiopaque materials may be incorporated at loadings toward the lower ends of these ranges (e.g., about 30% to about 50% by weight) and tungsten can be incorporated at relatively higher loadings (e.g., up to about 80% by weight).

The first, inner layer 10, as shown in FIG. 1, is generally tubular in form. The walls are, in certain embodiments, substantially uniform in thickness along the length of the tube and/or across the circumference of the tube. Layer 10 can have an average wall thickness, for example, of about 0.005″ to about 0.020″, e.g., about 0.005″ to about 0.015″, such as an average wall thickness of about 0.010″. It is understood that the thickness and/or uniformity of the walls of layer 10 can, in some embodiments, vary depending on whether the layer is in expanded (heat shrink) form or in used/shrunk form.

The second, outer layer 12, in some embodiments, comprises a fluoropolymer-based heat shrink tubing. The composition of the heat shrink tubing is not particularly limited. As an example, the fluoropolymer can be fluorinated ethylene propylene (FEP), polytetrafluoroethylene (PTFE), perfluoroalkoxy alkane (PFA), or copolymers, mixtures, or derivatives thereof. Layer 11 can, in some embodiments, consist essentially of one or more fluoropolymer(s).

In some embodiments, layer 12 comprises a peelable tubing. An example of a peelable tubing and manufacture of such is described in U.S. Pat. No. 10,434,222 to Roof et al., which is incorporated herein by reference in its entirety. By “peelable” is meant that the outer layer 12 can be readily removed from the construction, leaving layer 10 intact if desired, e.g., such that layer 12 can be used to position layer 10 while forming a medical device without becoming part of the final medical device into which it is incorporated. To facilitate peelability, the composition of layer 12 can be selected accordingly. In some embodiments, the layer can thus include a blend with other materials to provide peelability. In some embodiments, in addition to the fluoropolymer referenced above, layer 12 can comprise up to about 30% by weight of a filler and/or additive to aid in peelability.

As with layer 10, layer 12 is also typically tubular in form and the walls are, in certain embodiments, substantially uniform in thickness along the length of the tube and/or across the circumference of the tube. The thickness of second, outer layer 12 is not particularly limited and in some embodiments, layer 12 can have an average wall thickness, for example, of about 0.07″ to about 0.020″. It is understood that the thickness and/or uniformity of the walls of layer 12 can, in some embodiments, vary depending on whether the layer is in expanded (heat shrink) form or in used/shrunk form.

Advantageously, the outer layer 12 can have a higher melt point temperature than the inner layer 10. These values will necessarily vary, depending upon the exact composition of layers 10 and 12. As one, non-limiting example, in some embodiments, layer 10 comprises 72D Pebax with a melting point of around 174° C. and layer 12 comprises FEP with a melting point around 260° C.

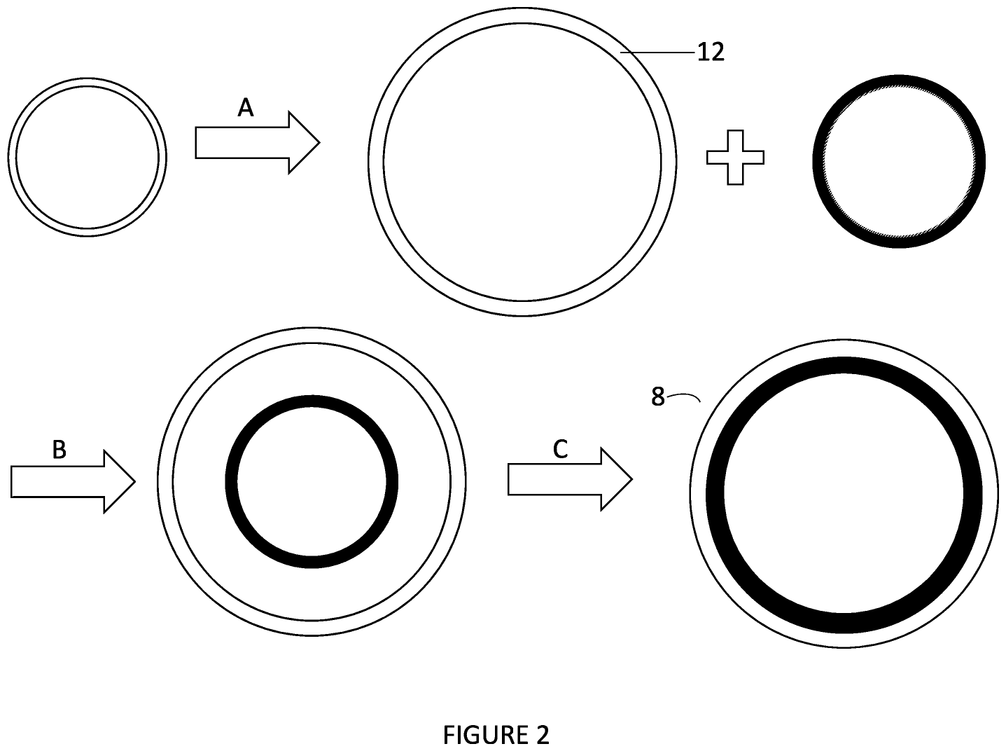

The construction 8 can be prepared, in part, as shown in FIG. 2. The depicted method includes, e.g., expanding a tube (step A) to give expanded layer 12, inserting the tubing for inner layer 10 within the expanded outer layer tubing (Step B), and expanding the inner layer within the outer layer (Step C), e.g., such that the outside surface of inner layer 10 is mated or touching the inner surface of the outer layer 12 as shown in the figures. The expansions can be done via conventional methods, e.g., using heat and pressure, e.g., to create a nested dual tube structure. The shrink ratio can vary as well. In some embodiments, the shrink ratio of an expanded inner diameter of the construction to a recovered inner diameter of the tube is greater than 1.2:1.

In some embodiments, construction 8 comprising the inner layer 10 and the outer layer 12 is manufactured as a dual layer heat shrink tubing. An example of a dual layer heat shrink tubing and manufacture thereof is described in U.S. Patent Application Publication No. 2021/0370581, which is incorporated herein by reference in its entirety. It is noted that, although the disclosure refers to the layers as an “inner” layer and an “outer” layer, the disclosure is not limited thereto. For example, the construction 8 can further comprise one or more additional layers, e.g., over layer 12 (such that layer 12 is not necessarily the “outer” layer of such construction). Similarly, the construction 8 can further comprise one or more additional layers, e.g., under (interior to) layer 10 (such that layer 10 is not necessarily the “inner” layer of such construction).

In various embodiments, the layer comprising the radiopaque filler advantageously comprises the radiopaque filler in the form of a material dispersed within the inner layer. The inner layer (at least) can ultimately be included as a component of a medical device (e.g., a catheter shaft). Such a construction provides the radiopaque marking as an integral component of the medical device (i.e., as an integral component incorporated within a layer, e.g., of a catheter shaft). This configuration distinguishes such constructions from constructions wherein radiopaque labels are simply applied to or otherwise associated with a medical device (e.g., as a traditional marker band). The disclosed construction ensures that the radiopaque marking does not move or come loose, e.g., during placement or during a medical procedure (as the radiopaque marking is dispersed throughout layer 10, which is an integral component of the medical device itself). As such, in some embodiments, the disclosed tube does not comprise any radiopaque components other than the radiopaque filler, i.e., no external radiopaque labels, e.g., no radiopaque labels (e.g., radiomarker bands) applied to or otherwise associated with the tube.

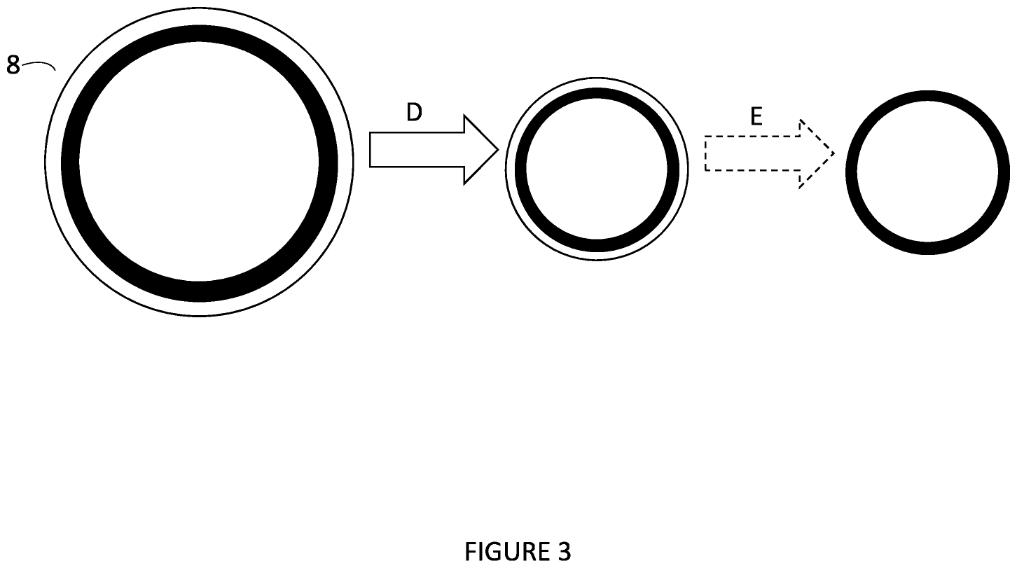

As such, in some embodiments, a medical device, such as a catheter assembly (e.g., catheter shaft) is provided which comprises at least a tube comprising radiopaque filler, e.g., as described above with respect to layer 10 of the construction used to assemble the medical device. For example, the tube corresponding to layer 10 can be present as the innermost layer of the medical device, e.g., the innermost layer (inner diameter) of the catheter shaft. In other embodiments, the tube corresponding to layer 10 can be present around another tube (e.g., a catheter liner tube) where the catheter liner tube forms the innermost layer (inner diameter) of the catheter shaft. An example method for providing such a medical device component is shown in FIG. 3, where construction 8 is positioned/placed (e.g., around a mandrel or around another component, e.g., a tube/liner, not shown); heat and/or pressure is applied (step D) to process construction 8 (which can, in some embodiments, result in shrink and/or reflow of outer layer 12 and/or inner layer 10). In some embodiments, as shown by the dotted arrow of Step E, the outer layer 12 is optionally removed from the medical device during assembly or following assembly (leaving only reflowed inner layer 10 within the device). For example, the outer layer can be a peelable tubing that is removed after reflow of the inner layer 10. In other embodiments, outer layer 12 can be retained within the medical device, such that the entire construction (e.g., in processed form, i.e., with components that have been shrunk and/or reflowed) is a component of the medical device.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### EXAMPLES

Aspects of the present invention are more fully illustrated by the following examples, which are set forth to illustrate certain aspects of the present invention and are not to be construed as limiting thereof.

### Example #1: Preparation of a Nested Pair

Two inputs were used in the construction of this example. One was a FEP heat shrink tube with a 0.256″ minimum Expanded ID, a 0.166″ maximum recovered ID, and 0.010″+/−0.002″ recovered walls. The other input was a Pebax® tube made with a compound comprising Pebax® 5533 SA01 MED with a 20 WT % Barium Sulfate loading and a blue colorant. The Pebax® tube had a 0.230″+/−0.0025″ ID and 0.010″+/−0.002″ Wall. The Pebax® tube was sealed on one end and then slid inside of the FEP Heat Shrink such that a small portion of the Pebax® tube extended beyond each end of the FEP Heat Shrink tube. The assemblage of 2 tubes was loaded into a vertical laminator with the open end of the Pebax® tube at the top and connected to an air supply system. Care was taken to ensure that only the Pebax® tube was connected to the air supply system and not the FEP Heat Shrink tube. The air supply system was opened and adjusted such that 75 psi was supplied to the inside of the Pebax® tube. The vertical laminator was then started and the heating nozzle traversed the sample at a rate of 2.5 mm/s while at a set point of 275° F. Once the cycle was completed the part was removed from the vertical laminator and the ends trimmed off.

### Example #2: Use of the Nested Pair of Example 1 in Creating a Catheter Shaft

An OD etched PTFE liner with a 0.002″ wall was slid over a tubular PTFE mandrel with a 0.222″ OD and the a braid of 0.002″ OD SS wire was applied on top of the OD Etched PTFE Liner. The part from EXAMPLE #1 was then slid over top of the braid covered liner. The assemblage was then loaded into a vertical laminator. The vertical laminator was started and the heating nozzle traversed the sample at a rate of 1.2 mm/s while at a set point of 480° F. The part was then removed from the vertical laminator. The outer FEP portion of the part was then removed with a skiving tool and the inner PTFE tubular mandrel removed resulting in a tube that is PTFE lined on the ID with a Blue Pebax® 5533SA01 MED with 20 WT % Barium sulfate outer layer and a SS wire braid between the 2 layers.

1. A tube, comprising:
an inner layer comprising thermoplastic material, wherein the thermoplastic is loaded with a radiopaque material; and
an outer layer comprising a fluoropolymeric material.

2. The tube of claim 1, wherein the thermoplastic material comprises one or more of Polyamide or Pebax (Poly-ether-block-amide).

3. The tube of claim 1, wherein the inner layer comprises the radiopaque material in an amount of about 5% to about 80% by weight, based on a total weight of the inner layer.

4. The tube of claim 1, wherein the inner layer comprises the radiopaque material in an amount of about 25% to about 50% by weight, based on a total weight of the inner layer.

5. The tube of claim 1, wherein the inner layer comprises the radiopaque material in an amount of about 30% to about 40% by weight, based on a total weight of the inner layer.

6. The tube of claim 1, wherein the radiopaque material is dispersed substantially uniformly throughout the inner layer.

7. The tube of claim 1, wherein the outer layer is peelable.

8. The tube of claim 1, wherein the radiopaque material comprises barium sulfate.

9. The tube of claim 1, wherein the radiopaque material comprises one or more of bismuth oxychloride, bismuth subcarbonate and tungsten metal powder.

10. The tube of claim 1, wherein the inner layer has an average wall thickness of about 0.010″.

11. The tube of claim 1, wherein the inner layer has an average wall thickness between 0.005″ and 0.020″.

12. The tube of claim 1, wherein the fluoropolymeric material of the outer layer comprises fluorinated ethylene propylene (FEP).

13. The tube of claim 1, wherein the fluoropolymeric material of the outer layer comprises polytetrafluoroethylene (PTFE).

14. The tube of claim 1, wherein the fluoropolymeric material of the outer layer comprises perfluoroalkoxy alkanes (PFA).

15. The tube of claim 1, wherein the outer layer has a higher melt point than the inner layer.

16. The tube of claim 1, wherein the inner and outer layers are in expanded form.

17. The tube of claim 1, wherein a shrink ratio of an expanded inner diameter of the tube to a recovered inner diameter of the tube is greater than 1.2:1.

18. A medical device manufactured using the tube of claim 1.

19. The medical device of claim 18, wherein the outer layer is removed.

20. A medical device comprising a tubing, wherein the tubing comprises a thermoplastic material loaded with a radiopaque material.

21. A medical device comprising the tube of claim 1.

22. A medical device comprising at least the inner layer of the tube of claim 1.

23. The medical device of claim 18, wherein the medical device is a catheter.

24. The medical device of claim 23, wherein the outer layer of the tube is removed during assembly of the medical device.
